(19) United States
(12) Patent Application Publication
(10) Pub. No.: US 2011/0262987 A1
(43) Pub. Date: Oct. 27, 2011

(54) Solubilization of Carbonaceous Materials and Conversion to Hydrocarbons and Other Useful Products

(76) Inventor: Robert A. Downey, Centennial, CO (US)

(21) Appl. No.: 13/091,730

(22) Filed: Apr. 21, 2011

Related U.S. Application Data
(60) Provisional application No. 61/342,916, filed on Apr. 21, 2010.
(60) Provisional application No. 61/378,590, filed on Aug. 31, 2010.

(51) Int. Cl. C12P 3/00; E21B 43/16; C09K 3/00; C01B 3/02; C07C 1/00; C11B 3/02

(52) U.S. Cl. 435/168; 585/733; 554/175; 252/182.12; 252/182.32; 423/648.1; 166/268

(57) ABSTRACT

Methods of producing useful products, such as hydrocarbons and other molecules that are useful as fuels, from carbonaceous materials, are disclosed. Such methods include obtaining a carbonaceous material, such as coal, from a deposit and treating the carbonaceous material with one or more chemicals, including acetic acid, salts of acetic acid, esters of acetic acid, hydroxides and peroxides, alone or in combination, to solubilize the material in preparation for further processing, such as bioconversion, to produce useful products, or solubilizing the carbonaceous material in a formation using the above-recited chemicals, removing the solubilized material from the formation and bioconverting it to produce useful products, or solubilizing the material using the above-recited chemicals and bioconverting at least a portion of the solubilized material in a formation followed by recovery of useful products from the formation.

This application claims priority of U.S. Provisional Application 61/342,916, filed 21 Apr. 2010, and U.S. Provisional Application No. 61/378,590, filed 31 Aug. 2010, the disclosures of which are incorporated herein by reference in their entirety.

### FIELD OF THE INVENTION

The present invention relates to the field of production of useful products, such as methane, carbon dioxide, gaseous and liquid hydrocarbons and other valuable products from carbonaceous materials, for example, coal, by solution mining of coal, direct introduction of chemicals into subterranean formations, and/or extraction of coal with further treatment to produce said chemicals, including use of anaerobic fermentation, such as utilizing non-indigenous microbial consortia.

### BACKGROUND OF THE INVENTION

Organic solvents such as carbon disulfide, tetrahydrofuran, pyridine, tetracyanoethylene, N-methyl-2-pyrrolidinone have been used separately and in combination to extract, for example, coal components. The extraction of coals with pyridine is also commonly performed in the coal industry.

When biomass is buried and subjected to pressure and temperature under increasingly anoxic conditions, the biomass is converted to peat, and then to low-rank coal, known as lignite. Lignite coal contains partially coalified plant materials, including lignins. As coalification increases, the oxygen content of the coal decreases, the carbon content increases, and the amount of lignin decreases.

Solubilization of the coal in the deposit itself would also be advantageous. For example, according to the United States Geological Survey, the coal-bearing basins of the United States contain deposits of more than 6 trillion tons of coal. The great majority of these coal deposits cannot be mined due to technical and economic limitations, yet the stored energy in these coal deposits exceeds that of U.S. annual crude oil consumption over a 2000-year period. Economical and environmentally sound recovery and use of some of this stored energy could reduce U.S. reliance on foreign oil and gas, improve the U.S. economy, and provide for improved U.S. national security.

About half of these coal deposits are of lignite or sub-bituminous rank and situated at depths of less than 3000 feet from the surface. These low-rank coal deposits are mined in several locations via strip mines, where overburden is removed, the coal is mined and the overburden is replace. The coal in these deposits have lower Btu content than bituminous coal, generally 5000 to 9000 Btu/pound, and a low market value, generally less than $11 per ton. The low Btu content of these coal deposits and low market value make them uneconomic to recover. Further, many of these coal deposits are situated geologically such that conventional surface or underground mining is impractical.

U.S. Pat. No. 3,990,513, incorporated by reference herein, discloses a process for the solution mining of coal. The patent discloses the use of solvents from the group consisting of phenanthrene, fluoranthene, pyrene and chrysene, heated to a temperature in the range of 250° C. to 400° C.

U.S. Pat. No. 4,501,445, incorporated by reference herein, discloses a process for in-situ hydrogenation of carbonaceous material, such as coal, oil shale and heavy oil deposits. The patent discloses a process for hydraulically fracturing and sealing a formation, followed by the injection of a liquid solvent stream and a gaseous hydrogen stream into the fractured formation, allowing reaction and conversion of the coal to lighter, hydrogenated products.

U.S. Pat. No. 5,120,430, incorporated by reference herein, discloses a process for the extraction of the organic portion of coal by the application of potassium hydroxide and select solvents.

Also relevant is co-pending U.S. patent application Ser. No. 12/965,285, entitled “Biogasification Of Coal To Methane And Other Useful Products” filed Dec. 10, 2010.

Previous studies have tested a range of chemical compounds for reaction with coal, and particularly for the solubilization of coal. Research has also been conducted on chemicals and processes for the liquifaction of coal. Focus has been predominantly on the chemical conversion of coal to hydrocarbon compounds that are directly utilized as a fuel or chemical product or chemical feedstock for the production of other chemicals or fuels. Coal can then be readily solubilized into carbonaceous material that can be metabolized by methanogenic consortia to methane, carbon dioxide and other hydrocarbons.

As the biomass undergoes the coalification process, bacteria and fungi can become entrained or enter the biomass deposit and are able to convert the carbon in the biomass or lignite or coal to methane, carbon dioxide and other products. The conversion of the coal is a slow and incomplete process.

The present invention above mentioned problems by providing methods for the treatment of coal and coal deposits to solubilize coal and in a preferred embodiment to treat coal to render coal more susceptible to conversion by bacteria and fungi to methane and other useful products. Such solubilization has been carried out either in the deposit itself (referred to as in situ solubilization) or on the coal itself following removal from a deposit (referred to as ex situ solubilization).

### BRIEF SUMMARY OF THE INVENTION

In one aspect, the present invention is directed toward methods of producing useful products from carbonaceous materials. Such methods include:

(i) contacting a carbonaceous material, such as coal, from a deposit, with one or more chemicals, to solubilize the material in preparation for further processing, such as bioconversion, to produce useful products, or

(ii) solubilizing the carbonaceous material in a formation using chemicals, removing the solubilized material from the formation and bioconverting it to produce useful products, or

(iii) solubilizing a carbonaceous material in a formation by adding chemicals to solubilize it and then and bioconverting at least a portion of the solubilized material while still in a formation using endogenous or exogenously added agents to produce useful products that can be recovered from the formation.

In separate embodiments, the solubilization chemicals include an organic acid (e.g., a carboxylic acid) of up to 4 carbon atoms or a benzoic acid, or a salt or ester of any of these acids. A preferred embodiment uses esters of acetic acid.

Chemicals and other agents can be added to a formation using a well, or well bores, that are then also available for removal of solubilized material or solubilized and bioconverted material.

Another aspect of the present invention is directed toward a composition comprising solubilized derivatives of a carbonaceous material, which derivatives are then available for bioconversion to hydrocarbons, such as methane, and other derivatives useful as fuels in energy production.

The geological formations useful for practicing the invention include subterranean formations, such as coalseams, shales, and oil sands, that contain useful carbonaceous materials.

The methods of the invention include processes wherein the chemicals are heated and then injected into a coal-bearing formation to solubilize coal contained therein. In a further example, such injection is carried out in combination with sonication to solubilize coal. Such methods are also available for treatment of a carbonaceous material after it has first been mined from a geological formation.

Where the carbonaceous material is coal, such coal is preferably a kind containing the largest amounts of fixed carbon and the smallest amounts of moisture and volatile matter.

### BRIEF DESCRIPTION OF THE DRAWINGS

FIG. 1 is a representative schematic plan view of a subterranean deposit of a hydrocarbon bed useful in explaining certain principles of the present invention. An ISBC process will include “patterns” of injection and production wells, and optionally include additional wells that may allow for the monitoring of the process—recording of pressure, temperature and flow data and sampling of formation fluids. The number and location of the monitoring wells would be carefully determined based upon a number of factors.

FIG. 2 is an isometric view of a portion of the deposit and related terrain of FIG. 1. In order to implement in-situ bioconversion of carbonaceous material, preferably coal, to methane (“ISBC”), a series of wellbores must be drilled into a coalseam and hydraulic connection established between the coalseam and the wellbore. Each injection well wellbore is then equipped so as to enable the injection of water, nutrients and chemicals from the surface into the coalseam, and devices that enable determination and recording of data such as pressure, temperature, and flowrate. Each producing well is then equipped so as to enable the production of water and generated gases and with devices that enable the determination and recording of data such as pressure, temperature, and flowrate of produced fluids and gases. Additional equipment is provided in the surface facilities that enable the sampling of injection and production fluids and gases for the a range of analyses that provide data on the microbial population in the fluids, the composition of the fluids and the nutrient composition in the fluids.

FIGS. 3a, 3b and 3c are isometric schematic views of the process for the solubilization of carbonaceous material and the recovery of the solubilized carbonaceous material, via the utilization of two or more wellbores extending from the surface to the carbonaceous material deposit. The plan view and cutaway shown illustrate the ISBC process. Water, nutrients and chemicals are injected into injection wells, and water and produced gases are produced from the offset producing wells. The amount and direction of the fluids flowing in the reservoir are optimized for the movement of nutrients into the coalseam, the movement of microbes, nutrients and generated gases in the coalseam, and the production of the water and gases. The adjustment of coalseam pressures at any point in the reservoir is made by adjusting the injection and production pressures in the injection and production wells.

FIG. 4 is a graph showing the amount of soluble carbon extracted from particles of a Powder River Basin coal source by the application of solubilization chemicals in a series of steps, in a Falcon tube test.

FIG. 5 is a graph showing the amount of soluble carbon extracted from particles of a Louisiana sub-bituminous coal source by the application of solubilization chemicals in a series of steps, in a Falcon tube test.

FIG. 6 is a graph showing the amount of soluble carbon extracted from coal by the application of solubilization chemicals in a series of steps, in a flow through tube test.

FIG. 7 is a process flow diagram of the anaerobic fermentation process utilized for the biological conversion of solubilized carbonaceous material to methane, carbon dioxide and other useful gases.

FIG. 8 is a graph depicting the amount of methane produced from solubilzed Powder River Basin coal in a two-stage anaerobic fermentation system operated in batch mode. The units of methane production are shown in standard cubic feet per ton of equivalent solubilized input coal.

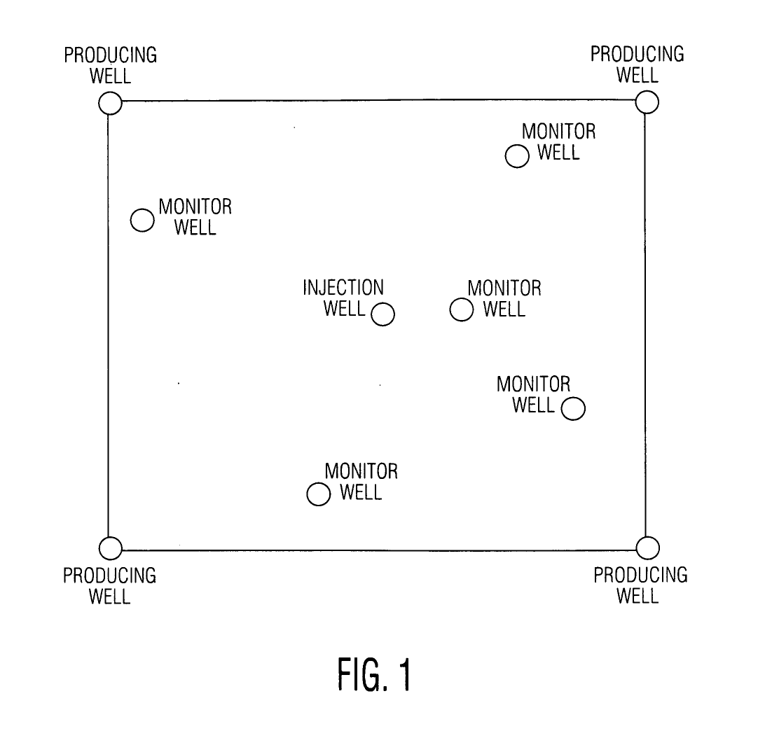

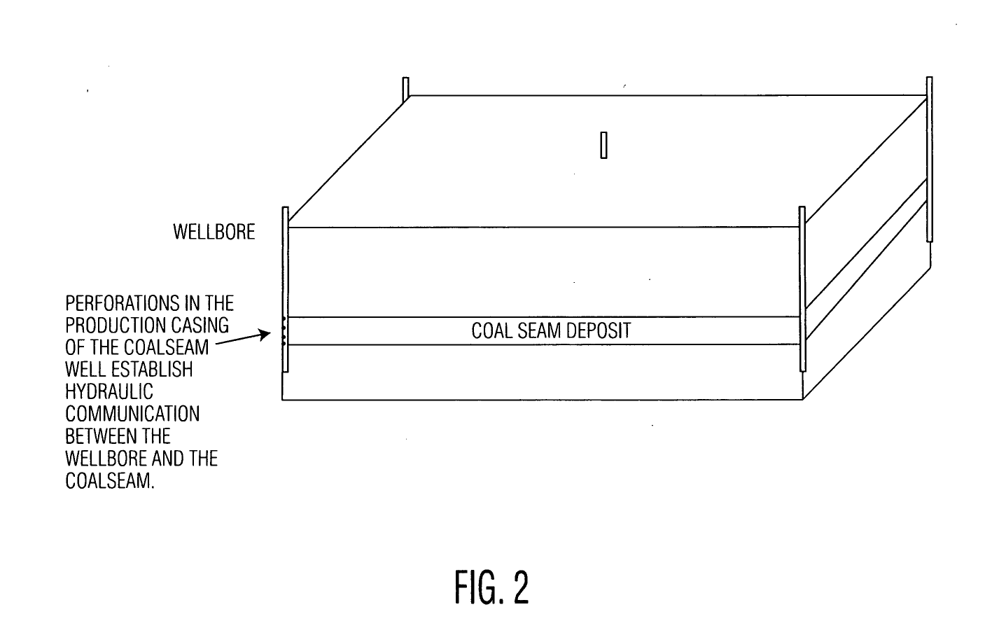

### DEFINITIONS

As used herein, “carbonaceous material” refers to materials containing the element carbon. These can include hydrocarbons and other materials, such as coal, and especially include naturally occurring deposits rich in carbon-containing compounds, such as hydrocarbons, both saturated and unsaturated. One example of such a material is coal.

As used herein, “coal” refers to any of the series of carbonaceous fuels ranging from lignite to anthracite. The members of the series differ from each other in the relative amounts of moisture, volatile matter, and fixed carbon they contain. Coal is comprised mostly of carbon, hydrogen and entrained water, predominantly in the form of large molecules having numerous double carbon bonds. Low rank coal deposits are mostly comprised of coal and water. Energy can be derived from the combustion of carbonaceous molecules, such as coal, or carbonaceous molecules derived from the solubilization of coal molecules. Of the coals, those containing the largest amounts of fixed carbon and the smallest amounts of moisture and volatile matter are the most useful. The lowest in carbon content, lignite or brown coal, is followed in ascending order by subbituminous coal or black lignite (a slightly higher grade than lignite), bituminous coal, semibituminous (a high-grade bituminous coal), semianthracite (a low-grade anthracite), and anthracite.

As used herein, the term “solubilizing” or “solubilized” refers to a process whereby the very large hydrocarbon molecules that comprise coal or other carbonaceous material are reduced to much smaller hydrocarbon molecules or compounds by the application of one or more chemicals that can cleave carbon bonds and other chemical bonds of coal molecules and react with the chemicals to form smaller hydrocarbon molecules that are then be biologically converted to methane, carbon dioxide and other useful gases.

Solubilization for the purposes of the invention means the conversion of a solid carbonaceous material, such as coal, to a form of carbon that is in solution with water, and more specifically a form of carbon comprised of compounds that are soluble in water and capable of passing through a 0.45 micron ter.

As used herein, the term “salts or esters of a carboxylic acid” means the conjugate base of such an acid, where the ion is formed by deprotonation of the acid. For acetic acid, the general formula is CH₃CO₂R, where R is an organic group.

As used herein, the term “acetate” refers to the salt wherein one or more of the hydrogen atoms of acetic acid are replaced by one or more cations of a base, resulting in a compound containing the negative organic ion of CH₃COO—. Said term also refers to an ester of acetic acid. In accordance with the invention, said salts or esters of acetic acid are optionally mixed with water. In one preferred embodiment, the salts or esters of acetic acid are used in admixture with water. It is to be appreciated that when such acetate salts are employed using a water solvent, some acetic acid may be formed (depending on the final pH) and will participate in the solubilization process. For purposes of the invention, a similar definition is to be understood where a salt of any other carboxylic acid, such as benzoic acid, is used for like purposes.

As used herein, the term “aromatic alcohol” means an organic compound having the formula ROH, wherein R is a substituted or unsubstituted aromatic group, which aromatic group may be a monocyclic ring or a fused ring. In one embodiment, the aromatic group R is unsubstituted. In another embodiment, R is substituted with one or more of a hydrocarbon group and/or an —OH group(s). In some embodiments, the —OH is present on the aromatic ring, or is present in a substituent of said ring or both.

As used herein, the phrase “microbial consortium” refers to a microbial culture (or natural assemblage) containing 2 or more species or strains of microbes, especially one in which each species or strain benefits from interaction with the other(s).

As used herein, the term “useful product(s)” refers to a chemical obtained from a carbonaceous material, such as coal, by solubilization and/or bioconversion and includes, but is not limited to, organic materials such as hydrocarbons, for example, methane and other small organics, as well as fatty acids, that are useful as fuels or in the production of fuels, as well as inorganic materials, such as gases, including hydrogen.

### DETAILED DESCRIPTION OF THE INVENTION

The present invention provides methods of treating a carbonaceous material, either in situ or ex situ, to solubilize at least a portion of the contents of the material and release components contained therein, which components are then recovered for further processing into fuels and other energy generating materials. The present invention also provides methods of producing such useful products from the solubilized carbonaceous materials through bioconversion processes.

The methods of the invention can be conveniently carried out in situ (where materials, i.e., chemicals and/or organisms, are added to a carbon-bearing formation, such as a coal seam, to effect a process of the invention), or ex situ (where carbonaceous material, such as coal, is first removed from a formation and then treated according to the methods of the invention), or so-called liquid mining of coal, as described in U.S. Pat. No. 3,990,513, which is hereby incorporated by reference, each incorporating a method of the invention.

The present invention provides methods of producing useful products, such as hydrocarbons like methane and other molecules that are useful as fuels, from carbonaceous materials, which methods include:

(i) obtaining a carbonaceous material, such as coal, from a deposit and treating the carbonaceous material with one or more chemicals, including a carboxylic acid, preferably acetic acid, salts of acetic acid, esters of acetic acid, as well as hydroxides and peroxides, alone or in combination, individually or sequentially, to solubilize the material in preparation for further processing, such as bioconversion, to produce energy yirlding products, or

(ii) solubilizing the carbonaceous material in a formation using the above-recited chemicals, removing the solubilized material from the formation and bioconverting it to produce useful products, such as fuels, or

(iii) solubilizing the material using the above-recited chemicals by adding these to a formation and bioconverting at least a portion of the solubilized material in the formation followed by recovery of the bioconverted products.

In one embodiment, a carbonaceous material, such as coal, obtained from a geological deposit is contacted with one or more chemicals, such as one or more of an organic acid (e.g., a carboxylic acid) of up to 4 carbon atoms or a benzoic acid, or a salt or ester of any of these acids, preferably acetic acid, including salts and esters of acetic acid, as well as a hydroxide and/or a peroxide, to effect solubilization of components of the carbonaceous material. The solubilized components are then further processed, such as by one or more bioconversion processes using microorganisms, to produce smaller organic molecules, such as hydrocarbons, like methane, useful in production of energy, fuels and the like.

In another embodiment, the carbonaceous material is solubilized in a geological formation and the resulting solubilized material recovered from the formation, followed by bioconversion to produce smaller organic molecules useful in production of energy, fuels and the like.

In a further embodiment, both solubilization and bioconversion are accomplished within a carbon-bearing formation and products are then removed in a form useful for energy production.

In accordance with the foregoing, the geological formations include mines, river beds, ground level fileds and the like, especially where these are rich in carbon-containing materials, for example, a coalseam.

In accordance with the invention, the carbonaceous materials are first solubilized, either in situ or ex situ, by contacting the material with one or more chemicals that break many of the chemical bonds that comprise the contained molecules and thereby serve to solubilize it. These chemicals, used either alone or in combination, are contacted with the carbon-containing material at selected concentrations, temperatures and steps in order to maximize the solubilization process.

The solubilization chemicals utilized in the present invention include peroxides, hydroxides, benzoic acids, C1-C4 carboxylic acids, preferably aliphatic acids, most preferably acetic acid, including salts or esters of any of these carboxylic acids, preferably esters such as acetates, that are employed individually, sequentially or in selected combinations and sub-combinations. In preferred embodiments, the latter chemicals are, or include, sodium hydroxide, hydrogen peroxide and/or ethyl acetate.

In one embodiment, the method includes contacting carbonaceous material that has been removed from a geological formation, preferably one rich in carbon-containing materials, with an organic acid (e.g., a carboxylic acid) of up to 4 carbon atoms or a benzoic acid, or a salt or ester of any of these acids, preferably acetic acid and/or one or more salts and/or one or more esters of acetic acid (i.e., one or more acetates) under conditions of temperature, pressure, and the like, that are effective to solubilize at least a portion of the carbonaceous material.

In one embodiment, solubilization is achieved by use of one or more esters of acetic acid, such as one or more of the acetates recited herein, with or without additional chemicals.

In one non-limiting example, carbonaceous material is contacted sequentially with one or more solubilization chemicals recited herein, which sequence comprises contacting the carbonaceous material with each of a peroxide, a hydroxide and a salt or ester of a carboxylic acid, preferably an acetate, especially an ester. Various combinations of these may also be used sequentially. Preferred agents include hydrogen peroxide, sodium hyroxide, and ethyl acetate. Sequential application of these chemicals is especially useful for in situ solubilization but may be used ex situ as well.

Other chemicals of similar composition may also be utilized. For example, potassium hydroxide in place of sodium hydroxide and/or a different acetate in place of ethyl acetate. The concentrations of these chemicals, as well as their relative volumes and the temperatures at which they are contacted with the coal, will vary depending upon a range of factors including the characteristics of the coal being solubilized and/or the conditions of any subterranean formation from which the coal is to be extracted.

In some embodiments, where the carbonaceous material is coal, said coal is lignite or any form or rank of coal, ranging from brown coal to anthracite, based on increasing carbon content. The lowest in carbon content, lignite or brown coal, is followed in ascending order by subbituminous coal or black lignite (a slightly higher grade than lignite), bituminous coal, semibituminous (a high-grade bituminous coal), semianthracite (a low-grade anthracite), and anthracite. All are useful in the methods of the invention.

In preferred embodiments, the contacting with one or more of the chemicals recited herein for solubilization is effected at temperatures in the range 0 to 300° C., including temperatures of 0 up to 200° C., preferably at a temperature of 10 to 200° C., or temperature ranges recited elsewhere herein.

In other preferred embodiments, the contacting with one or more of the chemicals recited herein for solubilization is effected at a variety of pH conditions that include pH ranges 2 to 12, 3 to 11, 5 to 10, and the like, or can lie in the acid or alkaline range, such as 1 to 6, 2 to 5, or 3 to 4, or in the range 8 to 13, or 9 to 12, or 10 to 11.

Useful combinations of temperature and pH are contemplated by the invention and those skilled in the art are believed well able to determine, without any undue experimentation, the conditions, or combinations of such conditions, best suited for treatment of any particular carbonaceous material or deposit. Use of these I combination with varying ranges of pressure are also contemplated.

In embodiments that utilize a salt or an ester of acetic acid, including, but not limited to, acetate salts and esters of alcohols and acetic acid, said salts or esters are optionally mixed with water. In one preferred embodiment, the salts or esters of acetic acid are used in admixture with water. Such acetate may also be an ester. Where such chemicals are introduced into a formation to solubilize at least a portion of the carbonaceous material therein, it may be advantageous to inject water ahead of the salt or ester.

Preferred esters of acetic acid useful in any of the methods of the invention include, but are not limited to, methyl acetate, ethyl acetate, propyl acetate, isopropyl acetate, n-butyl acetate, isobutyl acetate, amyl acetate, isoamyl acetate, hexyl acetate, heptyl acetate, octyl acetate, nonanyl acetate, decyl acetate, undecyl acetate, lauryl acetate, tridecyl acetate, myristyl acetate, pentadecyl acetate, cetyl acetate, heptadecyl acetate, stearyl acetate, behenyl acetate, hexacosyl acetate, triacontyl acetate, benzyl acetate, bornyl acetate, isobornyl acetate and cyclohexyl acetate.

It is appreciated that when such salts are employed using a water solvent, some acetic acid, or other carboxylic acid, will be formed (depending on the final pH) and is then free to participate in the solubilization process.

Another type of solvent that can be combined with an acetate or other ester of the invention is a phosphite ester. An ester of phosphite is a type of chemical compound with the general structure P(OR)₃. Phosphite esters can be considered as esters of phosphorous acid, H₃PO₃. A simple phosphite ester is trimethylphosphite, P(OCH₃)₃. Phosphate esters can be considered as esters of phosphoric acid. Since orthophosphoric acid has three —OH groups, it can esterify with one, two, or three alcohol molecules to form a mono-, di-, or triester. Chemical compounds such as esters of phosphite and phosphate, or an oxoacid ester of phosphorus, or a thioacid ester of phosphorus; or a mixture of an oxoacid of phosphorus and an alcohol, or a mixture of a thioacid of phosphorus and an alcohol, react with carbon-bearing molecules to break carbon bonds within the molecules and add hydrogen molecules to these carbon-bearing molecules. Such reaction yields a range of smaller carbon-bearing molecules, such as carbon monoxide, carbon dioxide and volatile fatty acids that are more amenable to bioconversion by methanogenic microbial consortia to methane and other useful hydrocarbons. The reaction products produced from reaction of the oxoacid ester of phosphorus or the thioacid ester of phosphorus, or a mixture of an oxoacid of phosphorus and an alcohol, or a mixture of a thioacid of phosphorus and an alcohol, with coal stimulates a methanogenic microbiological consortium in the subterranean formation to start producing, or increase production of, methane and other useful products.

In some embodiments, additional solvents can be combined with, or used in conjunction with, those already recited (i.e., organic acid (e.g., a carboxylic acid) of up to 4 carbon atoms or a benzoic acid, or a salt or ester of any of these acids, preferably acetic acid, salts or esters of acetic acid, as well as hydroxides and peroxides) in order to facilitate the solubilization process. Useful additional solvents include aromatic hydrocarbons, creosote and heavy oils. The preferred aromatic hydrocarbons include phenanthrene, chrysene, fluoranthene and pyrene, nitrogenous ring aromatics, for example, acridine and carbazole, as well as catechol and pyrocatechol, are also suitable as solvents in the processes of the invention. Aromatics such as anthracene and fluorene can also be used.

Additional solvents that can be used in conjunction with an organic acid, including a C1-C4 carboxylic acid, such as acetic acid, salts or esters of acetic acid, a benzoic acid, hydroxides and peroxides, include phosphorous acid, phosphoric acid, triethylamine, quinuclidine HCl, pyridine, acetonitrile, diethylether, acetone, dimethyl acetamide, dimethyl sulfoxide, tetrahydrothiophene, trimethylphosphine, HNO₃, EDTA, sodium salicylate, triethanolamine, 1,10-o-phenanthroline, sodium acetate, ammonium tartrate, ammonium oxalate, ammonium citrate tribasic, 2,3-dihydroxylbenzoic acid, 2,4-dihydroxylbenzoic acid, 3,4-dihydroxylbenzoic acid, 3,5-dihydroxylbenzoic acid, THF tetrahydrofuran.

A useful solvent includes any of the foregoing, as well as mixtures thereof, preferably a eutectic composition. Such mixtures can usefully be dissolved in a carrier liquid, for example, a heavy oil (such a mixture being no more than about 5% to 10% of the dissolved solvent). Such solvents are most useful when heated to temperatures in the range of 80 to 400° C., preferably 80 to 300° C., more preferably 100 to 250° C., and most preferably at least about 150° C. Temperatures higher than about 400° C. are less advantageous.

The present invention specifically contemplates facilitating bioconversion of carbon-bearing materials within subterranean formations to produce hydrocarbons, such as methane, by treating the subterranean formation with a solution of one or more of a C1-C4 carboxylic acid, such as acetic acid and/or a salt of acetic acid and/or an ester of acetic acid, or a benzoic acid or benzoate, preferably an ester, and also treating the formation with a solution containing at least one of an oxoacid ester of phosphorus or a thioacid ester of phosphorus; one or more aromatic alcohols; and one or more other chemicals selected from the group consisting of: hydrogen, oxoacids of phosphorus, salts of oxoacids of phosphorus, vitamins, minerals, mineral salts, metals, and yeast extracts.

In situ bioconversion of carbon-bearing subterranean formations to methane and other hydrocarbons, as well as carbon dioxide, is performed using indigenous or non-indigenous methanogenic consortia via the introduction of microbial nutrients, methanogenic consortia, or chemicals, utilizing a comprehensive mathematical model that fully describes the geological, geophysical, hydrodynamic, microbiological, chemical, biochemical, thermodynamic and operational characteristics of such systems and processes.

The amount of bioconverted products, and the rate of their production, is recognized herein as a function of several factors, including but not necessarily limited to, the specific microbial consortia present in a formation, such as a coalseam, the nature or type of the carbon-bearing (i.e., carbonaceous) formation, the temperature and pressure of the formation, the presence and geochemistry of the water within the formation, the availability and quantity of nutrients required by the microbial consortia to survive and grow, the presence or saturation of methane and other bioconversion products or components, and several other factors.

The rate of carbon bioconversion is proportional to the amount of surface area available to the microbes in the consortium, the population of the microbes and the movement of nutrients into the deposits and bioconverted products extracted from, or passing out of, the deposit as the deposit is depleted. The amount of surface area available to the microbes is proportional to the percentage of void space, or porosity, of the subterranean formation; and the permeability, or measure of the ability of gases and fluids to flow through the subterranean formation is in turn proportional to its porosity. All subterranean formations are to some extent compressible, i.e., their volume, porosity, and permeability is a function of the net stress upon them. Their compressibility is in turn a function of the materials, i.e., minerals, hydrocarbon chemicals and fluids, the porosity of the rock and the structure of the materials, i.e., crystalline or non-crystalline.

In accordance with the invention, bioconversion is effected by one or more bioconversion agents that include, but are not limited to, facultative anaerobes, such as those of the genus Staphylococcus, Escherichia, Corunebacterium and Listeria, acetogens, for example, those of the genus Sporomusa and Clostridium, and methanogens, for example, those of the genus Methanobacterium, Methanobrevibacter, Methanocalculus, Methanococcoides, Methanococcus, Methanocorpusculum, Methanoculleus, Methanofollis, Methanogenium, Methanomicrobium, Methanopyrus, Methanoregula, Methanosaeta, Methanosarcina, Methanophaera, Methanospirillium, Methanothermobacter, and Methanothrix. A more detailed list is provided below. The bioconversion agents can also be eukaryotes, such as fungi.

The bioconversion is operated under conditions effective to bioconvert the treated carbonaceous material and/or products obtained from it by treating with chemicals disclosed herein for solubilization. Useful bioconversion agents include facultative anaerobes, acetogens, methanogens and fungi as described elsewhere herein. Suitable bioconversion includes formation of hydrocarbons such as methane, ethane, propane; and carboxylic acids, fatty acids, acetate, carbon dioxide. Such bioconversion agents are useful in bioconversion of substrates solubilized either before or after removal from geological deposits.

In one preferred embodiment, coal is bioconverted by a combination of solubilization of coal by one or more of the solubilization chemicals disclosed herein, such as an acetate, or combination of an acetate with other agents, preferably either or both of a hydroxide and a peroxide, and bioconversion of the treated coal and/or coal solubilization product, using one or more chemicals and/or nutrients and/or vitamins and/or minerals recited herein to promote bioconversion of the treated coal and/or coal solubilization products. Such materials are employed as a supplement for growth and/or to enhance the bioconversion action of the organisms used as a bioconversion agent.

For example, U.S. Pat. No. 6,543,535 and U.S. Published Application 2006/0254765 disclose representative microorganisms and nutrients, and the teachings thereof are incorporated herein by reference. Suitable stimulants can also be included,

Bioconversion facilitating additives include major nutrients, vitamins, trace elements (for example, B, Co, Cu, Fe, Mg, Mn, Mo, Ni, Se, W, Zn as a non-limiting group) and buffers such as phosphate and acetate buffers). Suitable growth media can also be included. In practicing the invention, it may be necessary to first determine the nature of the microbial consortium present in the deposit, such as a coalseam, in order to determine the optimum growth conditions to be used as part of the inventive process.

Bioconversion of solubilized carbonaceous materials, such as solubilized coal, in situ is accomplished by a combination of bacteria, for example, one that includes two or more of facultative anaerobes, acetogens, methanogens and/or fungi, especially those of one or more of the genuses recited elsewhere herein. Such combination may be endogenously present in the deposit and/or is added to the deposit as part of the solubilization and/or bioconversion process. In specific embodiments, one or more nutrients, vitamins, minerals, metal catalysts and other chemicals are added to the coal deposit to promote the growth of the bacteria or fungi.

In accordance with the invention, such formation includes, but is not limited to, subterranean formations, such as coalseams, shales, and oil sands. One embodiment introduces into a subterranean carbonaceous formation one or more chemicals selected from a peroxide, a hydroxide, and an acetate, alone or in combination, thereby solubilizing the carbonaceous material in the formation and preparing it for further processing to produce fuels, or products readily converted into fuels, and the like.

With the injection of solubilization chemicals into a carbonaceous material-containing subterranean formation, such as a coalseam, the amount of solubilized carbonaceous materials produced, and the rate of such production, are a function of several factors, including but not necessarily limited to, the specific chemical compounds introduced to the carbonaceous subterranean formation, the concentration of the chemical compounds, the temperature of the chemical compounds, the order of application of the chemical compounds, the relative volumes of the chemical compounds, the introduction rate of the chemical compounds, the nature or type of the carbon-bearing formation, the temperature and pressure of the formation, the presence and geochemistry of the water within the formation, the presence or saturation of methane and other bioconversion products or components, and several other factors. Therefore the efficient solubilization of the carbonaceous material in the carbon-bearing subterranean formation requires optimized methods and processes for the delivery and dispersal of chemical compounds into the formation, the dispersal of chemical compounds across the surface area of the formation, the exposure of as much surface area of the formation to the chemical compounds, and the removal and recovery of the solubilized carbonaceous material and gases from the formation.

In one embodiment, the chemicals, which can include water or some other solvent, are introduced into said formation by conduits, such as one or more wellbores, extending from the surface to a carbonaceous subterranean deposit. These are oriented horizontally, vertically or at any other desired angle with respect to the surface and/or the subterraneous carbonaceous layer or formation. Such formation includes, but is not limited to, a coalseam, a shale, an oil sand or a heavy oil deposit.

In one embodiment, the conduits or wellbores are arranged in an array of patterns or configurations to displace injected chemicals into the subterranean formation and recover solubilized carbonaceous material. In separate embodiments, the solubilization chemicals are injected and/or the solubilized carbonaceous material is produced continuously or intermittently.

The methods of the invention also contemplate use of sonication during or after the treating or contacting with a chemical agent, which sonicating is optionally part of the solubilizing process or is used only to form a more uniform product that results from the treating or contacting.

In some embodiments, sonication may be employed together with the recited chemicals to achieve more uniform solubilization. Where sonication is used in conjunction with the solubilization of in situ carbonaceous material, such sonication can occur before, during or after introduction of solubilizing chemicals into the formation and is conveniently accomplished using, for example, a downhole sonication device.

In another embodiment, the subterranean formation is fracture stimulated prior to the injection of solubilization chemicals. Alternatively, the subterranean formation is fracture stimulated during injection of the solubilization chemicals by injection at rates and pressures sufficient to cause the formation to fracture.

In the embodiments of the invention, the solubilization products include carbonaceous materials in soluble or insoluble solid form, including gases.

The methods of the invention also contemplate concentrating the produced/recovered solubilized carbonaceous material, for example, by membrane separation, filtration, evaporation, or other suitable means.

The present invention also contemplates recycling or re-using water and/or solubilization chemicals used in the solubilization and/or concentration processes of the invention.

One embodiment of the invention includes determining or estimating the volumes and mass of subterranean formation, carbon content, porosity, fluid, and gases and solubilization chemicals and solubilized carbonaceous materials at any given time before, during and after applying the method according to the first and second embodiments.

A further embodiment includes determining the amount of carbon in the subterranean formation that is solubilized, at any given time before, during and after applying the method according to the one and second embodiments.

In a still further embodiment, one or more physical properties of the deposit comprise depth, thickness, pressure, temperature, porosity, permeability, density, composition, types of fluids and volumes present, hardness and compressibility. Knowledge of such properties is considered highly useful in determining the combination of chemicals to be used in the in situ solubilization process as well as any subsequent in situ bioconversion process.

In another embodiment, the operating conditions comprise one or more of injecting into the deposit: predetermined amounts of the solubilizing chemical solutions and predetermined amounts of water at predetermined flow rates.

In particular embodiments, the method of the invention takes advantage of the properties of the solubillizing chemical solutions include the concentrations, volumes, temperatures and delivery pressures and flowrates.

In one embodiment, the solubilized product is first dissolved in water and/or in particulate form. In another embodiment, at least one gaseous product is produced along with the solubilized carbon, wherein the process includes recovering the at least one gas from the deposit.

One or more separate embodiments include recovering the solubilized carbonaceous material and at least one gas from the deposit and a simulation includes dividing the deposit into at least one grid of a plurality of three dimensional deposit subunits, and predicting the amount of recovery of the solubilized carbonaceous material and at least one gas from one or more subunits.

One or more other embodiments include dividing the subterranean carbonaceous deposit into a grid of a plurality of three dimensional subunits, selecting the subunit exhibiting an optimum amount of solubilized carbonaceous product to be recovered and then recovering the solubilized product from that selected subunit.

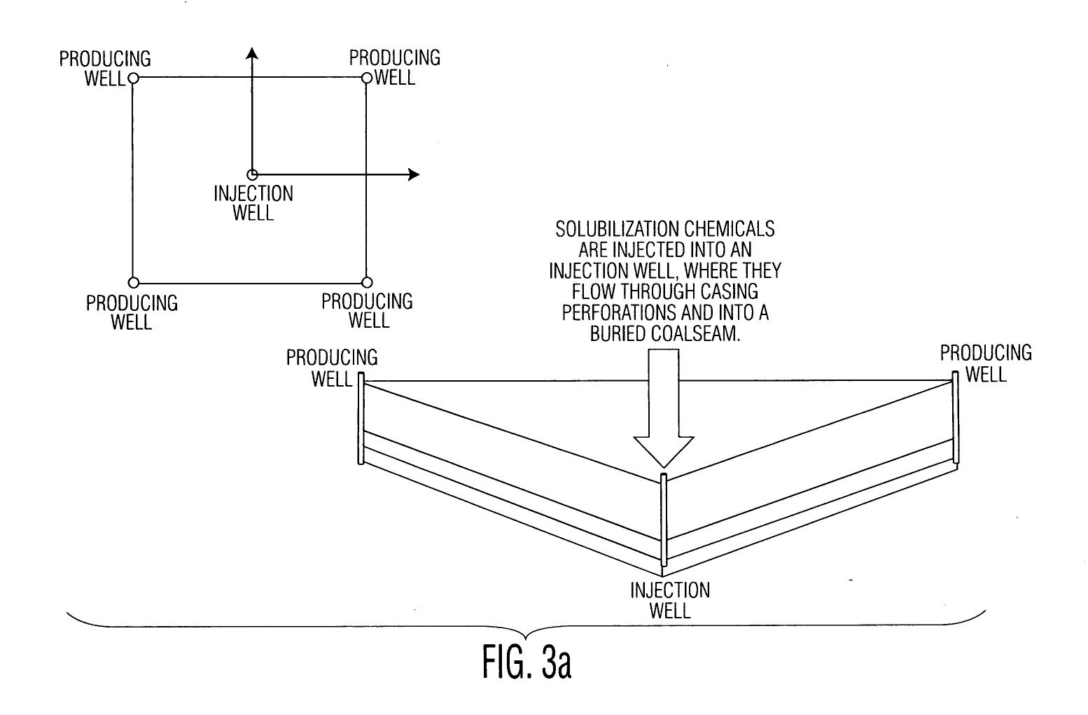

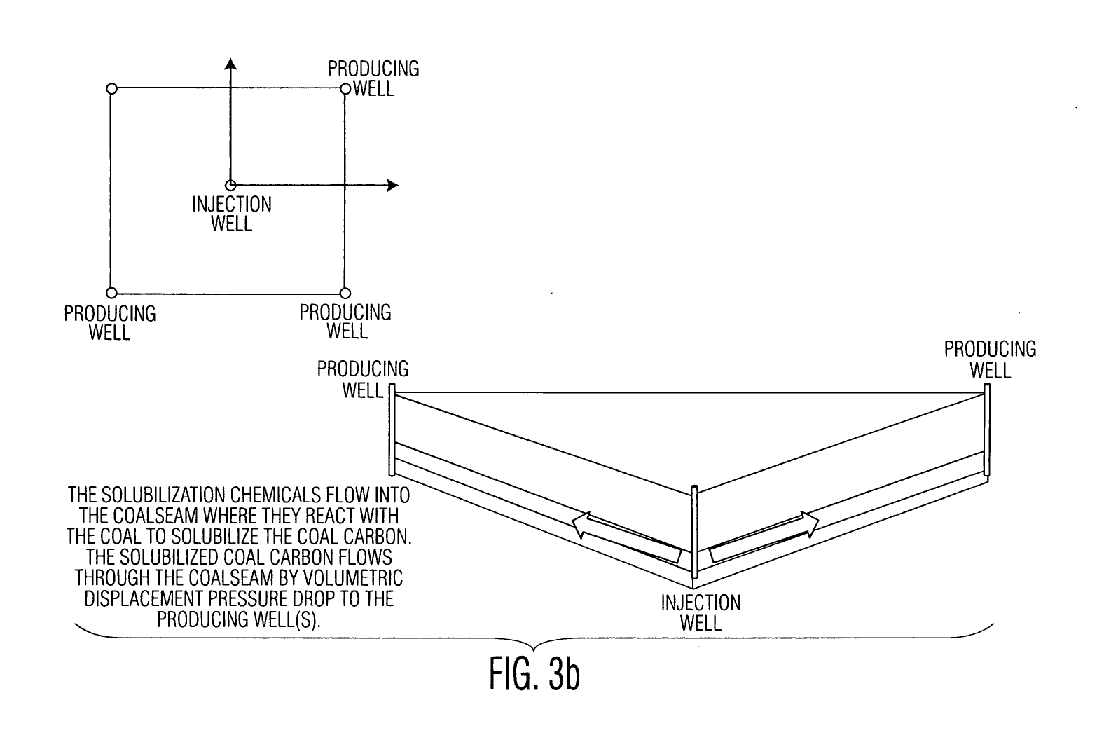

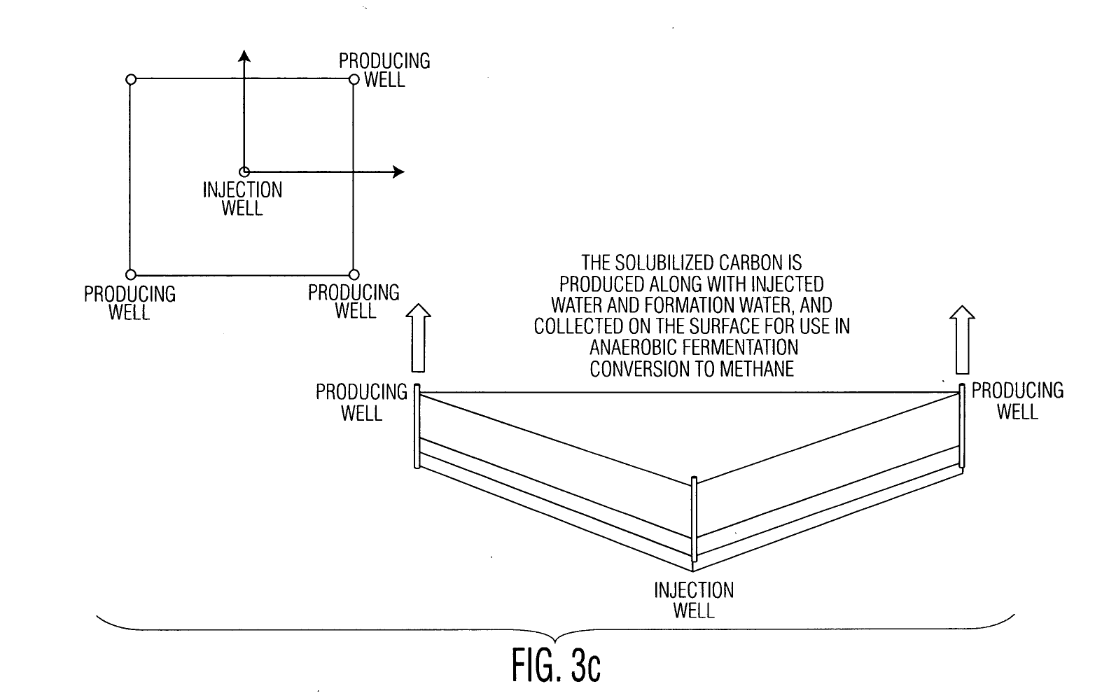

The methods of the invention specifically contemplate recovering the solubilized carbonaceous product from the deposit wherein the simulation includes dividing the deposit into at least one grid of a plurality of three dimensional deposit sectors, and predicting the amount of recovery of the solublized carbonaceous material and at least one gas from one or more sectors, and determining the flow of the solubilized carbonaceous material and gaseous product from sector to adjacent sector. In one specific example, the general method of the invention comprises the steps of FIG. 3.

In a preferred embodiment, where the solubilization chemicals include at least two of a peroxide, a hydroxide and an acetate, more preferably where all three are utilized, the chemicals are contacted with a subterranean deposit, layer or formation either as a mixtures or sequentially, such as a sequence of injections of said chemicals. When added as a mixture, the chemicals are added together as a single composition or are added in sequence so that the mixture forms in situ. When added in sequence, each injection is optionally separated from the one before or after by injection of a suitable solvent, for example, water.

For example, one embodiment includes injecting the peroxide, followed by injecting the hydroxide, followed by an acetate, each such injection separated by an injection of a volume of water. Several non-limiting embodiments are provided in the examples with results shown in the figures.

In one embodiment, the solubilization chemicals comprise at least one peroxide, at least one hydroxide and at least one ester, preferably an acetate, together with additional chemicals, either by separate injection or injection together with a peroxide, hydroxide or acetate.

In accordance with the invention, the solubilized carbonaceous material is commonly recovered, for example, via one or more of the conduits or wellbores used to introduce the solubilization chemicals. Such recovery can also be by use of additional conduits or wellbores formed for that purpose and different from those used to introduce the solubilization chemicals. The same or separate conduits or wellbores are formed for the purpose of testing the amount of material in the formation and/or monitoring the progress of the solubilization process.

In one embodiment of the invention, the solubilizing chemicals include at least one hydroxide. In preferred embodiments, the hydroxide is a hydroxide of sodium, potassium, aluminum, calcium, magnesium, ammonium, copper, or iron, with sodium hydroxide being especially preferred. Such hydroxide is present in a concentration of 0.01% to 50%, preferably 0.1% to 40%, more preferably 1% to 30%, or 1.5% to 20%, or 2% to 10%, most preferably 2.5% to 5%, with about 3%, 3.5% and 4% 4.5% being most preferred concentrations.

In embodiments where the solubilization chemicals include a peroxide, the preferred agent is hydrogen peroxide. Such peroxide is preferably added in a concentration of 0.01% to 50%, preferably 0.1% to 40%, more preferably 1% to 30%, or 1.5% to 20%, or 2% to 10%, most preferably 2.5% to 5%, with about 3%, 3.5% and 4% being most preferred concentrations.

In one embodiment, the peroxide is combined with another reagent, such as an iron catalyst, for example, iron(II) sulfate. Such a combination is commonly referred to as Fenton's reagent. Such peroxide is added in a concentration of 0.01% to 50%, preferably 0.1% to 40%, more preferably 1% to 30%, or 1.5% to 20%, or 2% to 10%, most preferably 2.5% to 5%, with about 2.5%, 3%, 3.5% and 4% being most preferred concentrations.

Such chemicals are especially useful when heated to temperatures in the range of 10° C. to 250° C., preferably 70° C. to 200° C., more preferably 70° C. to 150° C., and most preferably 70° C. to 100° C. Temperatures higher than about 250° C. are less advantageous.

In one embodiment, the treating or contacting is effected at a variety of pressure conditions that include atmospheric pressure, above atmospheric pressure, or below atmospheric pressure. For example, in treating coal deposits in situ, the pressure is be the pressure prevailing in the deposit or at an elevated pressure by controlling the pressure at which liquid is introduced into the well.

In one such embodiment, the solubilizing chemicals are introduced into the subterranean formation under a pressure of 0 psig to 5000 psig per foot of depth from the surface to the depth of the subterranean formation, preferably wherein said pressure 0.44 psig and 0.7 psig.

The invention contemplates that such conditions of solubilization are not mutually exclusive but that advantageous combinations of temperature, pressure and concentrations of the different solubilizing chemicals are well within the skill of those in the art to produce and yet be fully within the boundaries of the invention.

In one preferred embodiment, the solubilization chemicals are hydrogen peroxide, sodium hydroxide and ethyl acetate.

In embodiments where the recovered solubilized carbonaceous material is contacted with an anaerobic fermentation system, such systems may be of varying configuration, including one-stage, two-stage and multi-stage fermentation systems for the bioconversion of the solubilized carbonaceous material into a gas, for example, where the gas is methane, carbon dioxide, a higher hydrocarbon or some other useful product, depending on the fermentation reagents employed.

Transport of the solubilized carbonaceous material by truck, rail or pipeline from the point of extraction to a location for anaerobic fermentation bioconversion is specifically contemplated by the invention and such fermentation need not be conducted at or near the site of solubilization.

Such fermentation can convert the solubilized carbonaceous materials into suitable organic acids, carboxylic acids, acetates and esters in a fermentation system and can employ indigenous or non-indigenous microbial consortia. In one embodiment, the solubilized carbon is converted to methane, carbon dioxide and other useful products by indigenous or non-indigenous methanogenic consortia.

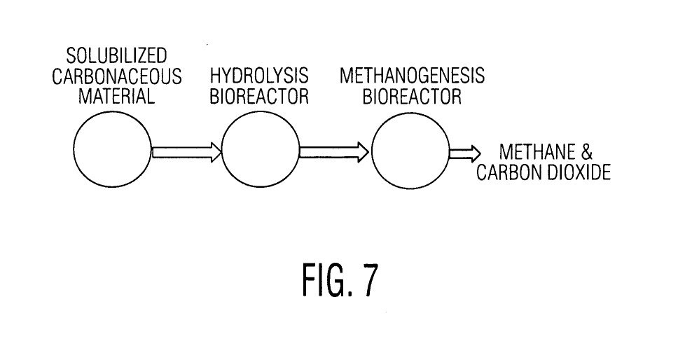

Microbial methanogenic consortia are utilized in anaerobic fermentation systems under optimized conditions of pressure, temperature and other factors to maximize the conversion of the solubilized carbon to methane, carbon dioxide and other useful hydrocarbons. Such a process can comprise introducing the solubilized carbonaceous materials into a first hydrolysis reactor followed by a second methanogenesis reactor to produce methane and carbon dioxide as shown in FIG. 7. Other hydrocarbons are produced according to a similar process.

In one embodiment, the biogasification reactor incorporates a material, or materials, having a high surface area to volume ratio, in order to serve as a surface for methanogenic bacterial culture attachment and growth.

Any active hydrolytic or methane producing mesophilic or thermophilic anaerobic digestion system can be used to produce useful products from solubilized carbonaceous materials formed in accordance with the methods of the present invention.

In one embodiment, hydrogen-producing anaerobic systems utilize microorganisms from the Clostridium species. For example, the Clostridium species can include, but are not be limited to, C. thermolacticum, C. thermohydrosulfuricum, C. thermosucinogene, C. butyricum, C. botulinum, C. pasteurianum, C. thermocellum and C. beijirincki. In a different embodiment, hydrogen-producing anaerobic systems utilize microorganisms from the Lactobacillus and/or the Eubacteria species. In non-limiting examples, the Lactobacillus species is a Lactobacillus paracasel, and/or the Eubacteria species is a Eubacteria aerogenes.

Preferred hydrolytic organisms include Clostridium, Bacteroides, Ruminococcus, Acetivibrio, Lactobacillus and other Firmicutes and Proteobacteria.

Methane-producing anaerobic systems utilizing acid forming bacteria and methane-producing organisms are well known and are readily employed to produce methane from sewage sludge or from brewery waste. These are specifically contemplated for use in the present invention. A review of the microbiology of anaerobic digestion is set forth in “Anaerobic Digestion, 1. The Microbiology of Anaerobic Digestion,” by D. F. Toerien and W. H. J. Hattingh, Water Research, Vol. 3, pages 385-416, Pergamon Press (1969).

Suitable acid forming species include species from genera such as, but not limited to, Aerobacter, Aeromonas, Alcaligenes, Bacillus, Bacteroides, Clostridium, Escherichia, Klebsiella, Leptospira, Micrococcus, Neiseria, Paracolobacterium, Proteus, Pseudomonas, Rhodopseudomonas, Rhodobacter sphaeroides, Rubrobacter species, Erythrobacter litoralis, Jannaschia sp., Rhodopirellula baltica, Sarcina, Serratia, Streptococcus and Streptomyces. Also of use in the present invention are microorganisms which are selected from the group consisting of Methanobacterium oinelianskii, Mb. Formicium, Mb. Sohngenii, Methanosarcina barkeri, Ms. Acetovorans, Ms. Methanica and Mc. Mazei, Methanobacterium thermoautotrophicus, Methanobacterium bryantii, Methanobrevibacter smithii, Methanobrevibacter arboriphilus, Methanobrevibacter ruminantium, Methanospirillum hungatei, Methanococcoides buntonii, Methanococcus vannielli, Methanothrix soehngenii Opfikon, Methanothrix sp., Methanosarcina mazei, Methanosarcina thermophila and mixtures thereof.

Preferred methanogenic organisms include Methanobacteriaceae, Methanosarcinaceae, Methanosaetaceae, Methanocorpusculaceae, Methaanomicrobiaceae and other archaea organisms.

Other useful microorganisms and mixtures of microorganisms are known to those of skill in the art. For example, U.S. Pat. No. 6,543,535 and U.S. Published Application 2006/0254765 disclose representative microorganisms and nutrients, and the teachings thereof are incorporated by reference. Suitable stimulants can also be included,

A wide variety of substrates are utilized by methane producing bacteria but each species is currently believed to be characteristically limited to the use of a few compounds. Therefore, several species of methane producing bacteria can be required for complete fermentation of materials recovered according to the invention. For example, the complete fermentation of valeric acid requires as many as three species of methane producing bacteria. Valeric acid is oxidized by Mb. Suboxydans to acetic and propionic acids, which are not attacked further by this organism. A second species, such as Mb. Propionicum, can convert the propionic acid to acetic acid, carbon dioxide and methane. A third species, such as Methanosarcina methanica, is required to ferment acetic acid.

It is understood that all embodiments described herein are given by way of illustration and not limitation and that one of ordinary skill may make modifications to the disclosed embodiments. For example, while one set of solubilization chemicals and/or concentrations is described, there may be any number of such concentrations in a given implementation and according to a given hydrocarbon formation. It is intended that the scope of the invention be determined in accordance with the appended claims.

### Example 1

### Solubilization of Carbonaceous Material

The method for the solubilization of carbonaceous material from coal was determined in a series of laboratory tests. Samples of coal were obtained from three different sources, the Caballo coal mine and a shallow coalbed methane well in the Powder River Basin of Wyoming, and from a wellbore drilled near Columbia, La. In the first series of tests, pieces of coal approximately 0.25 inches in diameter and total weight of approximately 5 grams were placed in falcon tubes were treated with 10 ml of hydrogen peroxide at 3% volume concentration was added to the falcon tube for a period of 24 hours at 25° C. The fluid was decanted, and then 10 ml of 50 mM molar sodium hydroxide heated to 90C was added to the tube for a period of 60 minutes. The fluid was decanted, and then 10 ml of 5% volume ethyl acetate heated to 75° C. was added to the tube for a period of 60 minutes. The fluid was decanted. This sequence of chemical addition and decanting was continued until 20 sequences were completed. The decanted fluids were analyzed for solubilized carbon content. The remaining coal solids were analyzed for mass and residual carbon content.

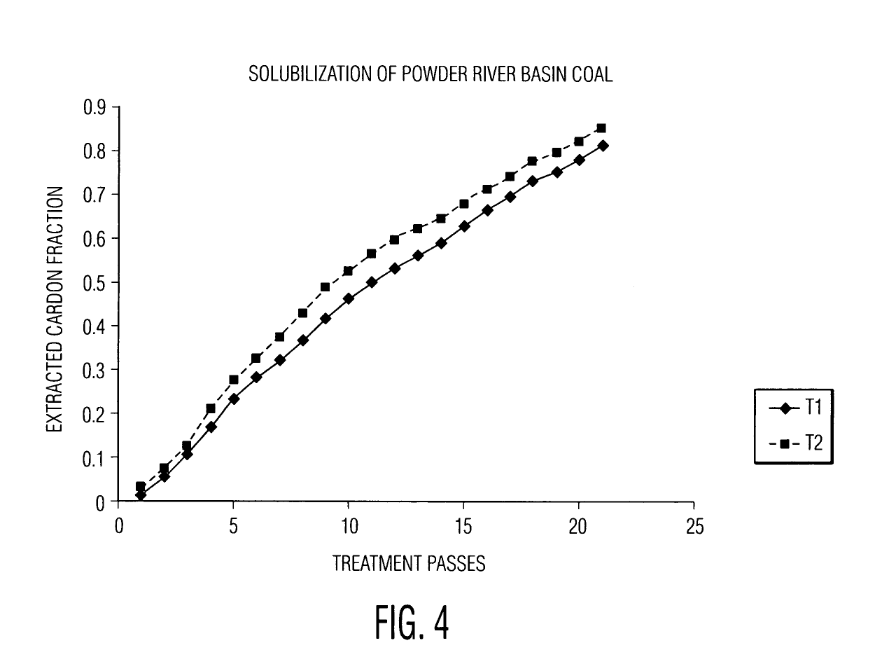

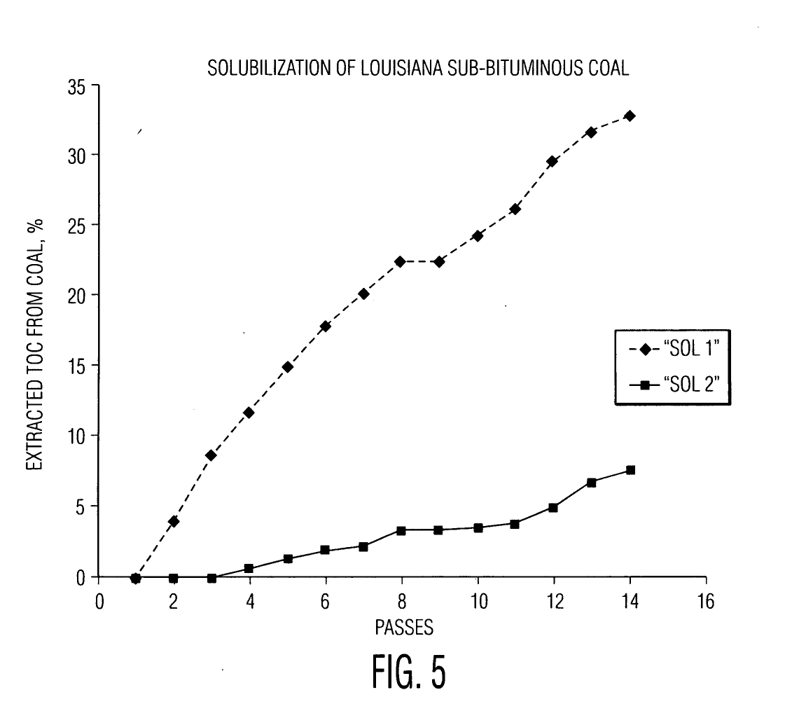

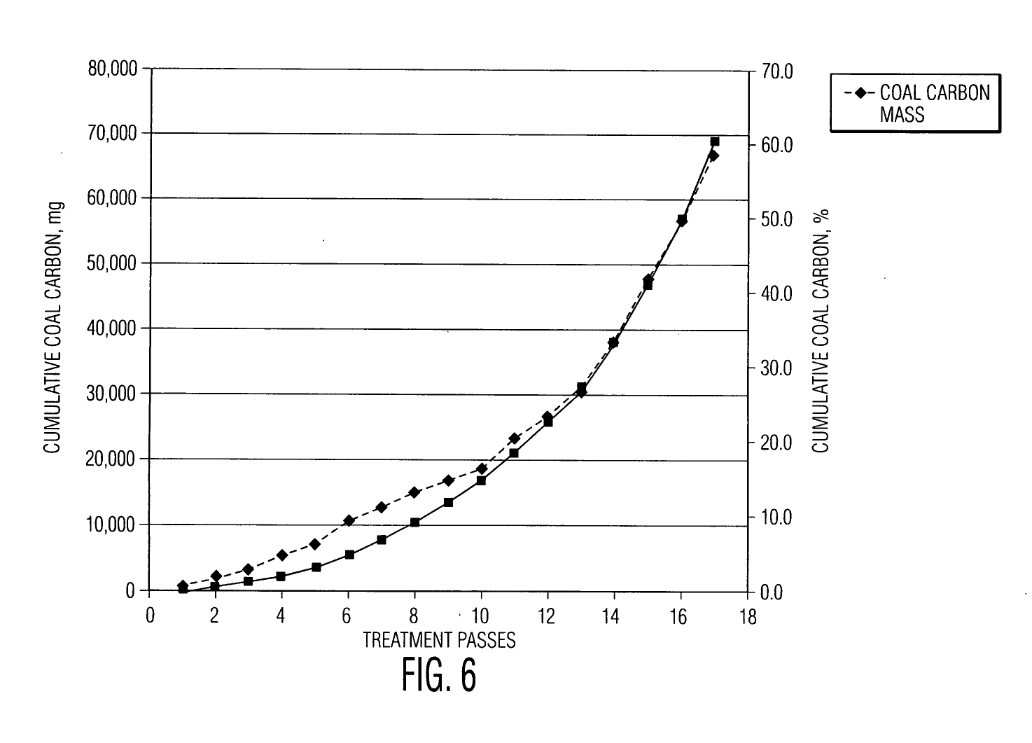

FIGS. 4 and 5 are graphs showing the amount of carbon solubilized from the coal in each solubilization step, for the two Powder River Basin coal samples tested, and for the Loisiana coal sample, respectively.

### Example 2

### Solubilization of Carbonaceous Material

A second test was conducted on a sample of coal derived from the North Antelope Rochelle coal mine in the Powder River Basin of Wyoming. In this test, pieces of coal of varying size but not smaller than 0.25 inches in diameter were placed into a stainless steel tube 2 inches in internal diameter and 26 inches long. Formation water was added to the tube to fill up all void spaces between the coal pieces. The tube ends were capped and fitted with ports and valves to enable the introduction and recovery of fluids into the tube. The tube was mounted vertically in a stand and connected to a pump, and the apparatus was fitted with instruments to measure pressure, flow and temperature into and out of the tube. Approximately 300 ml of 0.88 molar hydrogen peroxide was pumped into the tube, followed by 300 ml of formation water. The time during which the hydrogen peroxide was pumped and then allowed to remain in the tube prior to the injection of formation water was 144 minutes. The time during which the formation water was pumped and then allowed to remain in the tube was 30 minutes.

Following the pumping of the formation water into the tube, 300 ml of 0.05 molar sodium hydroxide was pumped into the tube, followed by 300 ml of formation water. The time during which the sodium hydroxide was pumped and then allowed to remain in the tube prior to the injection of formation water was 60 minutes. The time during which the formation water was pumped and then allowed to remain in the tube was 30 minutes. Following the pumping of the formation water into the tube, 300 ml of 0.5% molar ethyl acetate heated to 90° C. was pumped into the tube, followed by 300 ml of formation water. The time during which the ethyl acetate was pumped and then allowed to remain in the tube prior to the injection of the formation water was 60 minutes. The time during which the formation water was pumped and then allowed to remain in the tube was 30 minutes. At the time of each chemical and formation water injection into the tube, fluids displaced by the injection were collected from the discharge port on the opposite end of the tube and tested for composition of solubilized carbon and injected chemicals. The process of chemicals and formation water injection in identical sequences and volumes was continued until twenty complete cycles of chemicals and formation water had been pumped into the tube, and samples collected from the discharge port of the tube.

FIG. 5 is a graph depicting the amount of solubilzed carbon produced by the solubilization process as a percentage of the total initial carbon content of the coal. At the completion of the pumping process, the volume of the coal and its composition were determined, along with the volume and composition of the fluids remaining in the tube. The solubilized carbon content of the fluids was determined using UV-Vis spectrophotometric methods and confirmed using liquid chromatography-mass spectrometric methods.

### Example 3

### Anaerobic Fermentation

A 67 gram sample of Powder River Basin coal solubilized by the chemical solubilization steps described above was introduced in small batch increments into a two-stage anaerobic fermentation system to determine the extent to which the solubilized carbonaceous material could be converted to methane, carbon dioxide and other gases. Over a 27-day period, the solubilized carbon was converted to approximately 52,000 ml of methane, or approximately 25,000 standard cubic feet per ton equivalent, and 24,000 ml of carbon dioxide.

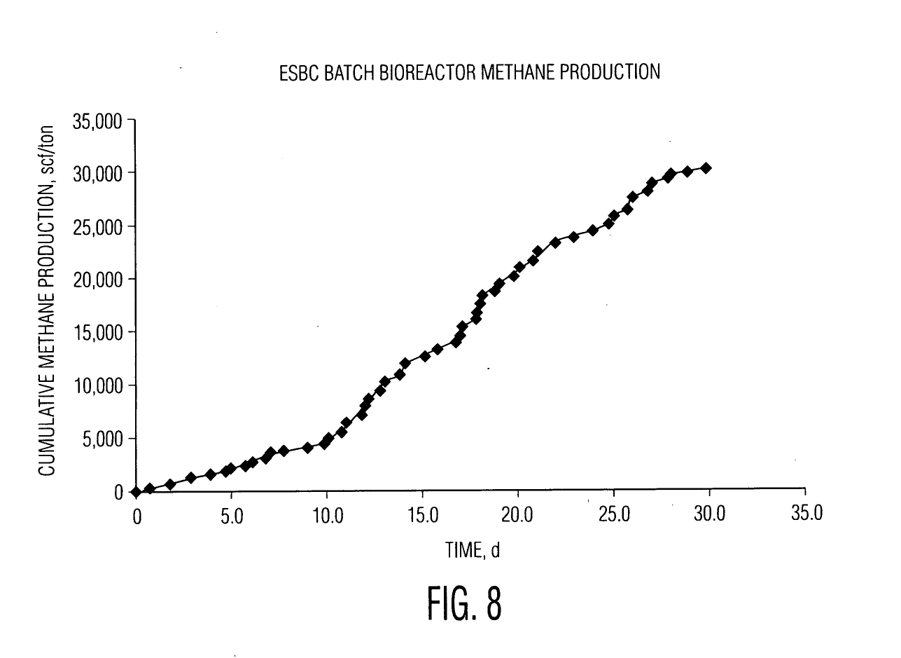

FIG. 8 depicts the amount of methane produced in the anaerobic fermentation system from the solubilized coal, measured in standard cubic feet per ton of input coal, over a 30-day period. Nearly all of the solubilized coal carbon was converted to methane and minor amounts of carbon dioxide in the anaerobic fermentation system.

### Example 4

### Solubility of Lignite in C₄H₈O₂

10 g of lignite was ground to approximately 250 micron size and sieved, then mixed with 50 ml of a 25% percent solution of ethyl acetate C₄H₈O₂ in water and heated at 90° C. for 2 hrs. The pressure was 14.7 psia and the pH was 7. The sample was found to be 93.5% soluble in C₄H₈O₂/water. A similar sample of lignite was found to be only 12% soluble in pyridine when treated under the same conditions.

What is claimed is:

1. A method of treating a carbonaceous material, comprising:
contacting a carbonaceous material with one or more solubilization chemicals selected from a carboxylic acid of up to 4 carbon atoms or a benzoic acid, or a salt or ester of any of these acids, thereby solubilizing at least a portion of the carbonaceous material.

2. The method of claim 1, wherein the carbonaceous material is coal.

3. The method of claim 2, wherein the coal is selected from the group consisting of lignite, brown coal, sub-bituminous coal, bituminous coal, anthracite, and combinations thereof.

4. The method of claim 1, wherein said contacting is carried out at a temperature of 0 to 300° C.

5. The method of claim 1, further comprising treating the at least a portion of the carbonaceous materials with one or more bioconversion agents during or after said contacting with said member.

6. The method of claim 5, further comprising adding nutrients, vitamins, minerals, and metal catalysts before or during the contacting.

7. The method of claim 5, wherein the bioconversion agent is at least one member selected from the group consisting of facultative anaerobes, acetogens, methanogens and fungi.

8. The method of claim 1, wherein said contacting further includes contacting with a solvent selected from the group consisting of phenanthrene, chrysene, fluoranthene and pyrene, a nitrogenous ring aromatic, anthracene, fluorene and combinations of any of these.

9. The method of claim 1, wherein said contacting further includes contacting with a solvent selected from the group consisting of phosphorous acid, phosphoric acid, a phosphite ester, triethylamine, quinuclidine pyridine, acetonitrile, diethylether, acetone, dimethyl acetamide, dimethyl sulfoxide, tetrahydrothiophene, trimethylphosphine, HNO3, EDTA, sodium salicylate, triethanolamine, 1,10-o-phenanthroline, sodium acetate, ammonium tartrate, ammonium oxalate, ammonium citrate tribasic, 2,3-dihydroxylbenzoic acid, 2,4-dihydroxylbenzoic acid, 3,4-dihydroxylbenzoic acid, 3,5-dihydroxylbenzoic acid, THF—tetrahydrofuran.

10. The method of claim 1, wherein said solubilization chemical is an ester of acetic acid.

11. The method of claim 10, wherein said ester of acetic acid is a member selected from the group consisting of methyl acetate, ethyl acetate, propyl acetate, isopropyl acetate, n-butyl acetate, isobutyl acetate, amyl acetate, isoamyl acetate, hexyl acetate, heptyl acetate, octyl acetate, nonanyl acetate, decyl acetate, undecyl acetate, lauryl acetate, tridecyl acetate, myristyl acetate, pentadecyl acetate, cetyl acetate, heptadecyl acetate, stearyl acetate, behenyl acetate, hexacosyl acetate, triacontyl acetate, benzyl acetate, bornyl acetate, isobornyl acetate and cyclohexyl acetate.

12. A method of solubilizing a carbonaceous material in a subterranean formation, comprising introducing into a subterranean carbonaceous formation one or more solubilization chemicals selected from a peroxide, a hydroxide, and an ester or salt of a C1-C4 carboxylic acid or benzoic acid, thereby solubilizing at least a portion of the carbonaceous material in said formation.

13. The method of claim 12, wherein said chemicals are introduced into said formation by one or more conduits or wellbores, extending from the surface to a carbonaceous subterranean deposit.

14. The method of claim 12, wherein said chemicals are injected sequentially, each said injection being separated by a volume of water.

15. The method of claim 14, wherein said chemicals are injected in the order peroxide, hydroxide and ester or salt of a C1-C4 carboxylic acid or benzoic acid.

16. The method of claim 12, further comprising recovering the solubilized carbonaceous material.

17. The method of claim 16, further comprising the step of contacting said recovered solubilized carbonaceous material with an anaerobic fermentation system for the bioconversion of said solubilized carbonaceous material into a gas.

18. The method of claim 15, further comprising recovering the solubilized carbonaceous material.

19. The method of claim 18, further comprising the step of contacting said recovered solubilized carbonaceous material with an anaerobic fermentation system for the bioconversion of said solubilized carbonaceous material into a gas.

20. The method of claim 12, wherein said subterranean formation is a coalseam, a shale, an oil sand or a heavy oil deposit.

21. The method of claim 12, wherein said hydroxide is a hydroxide of sodium, potassium, aluminum, calcium, magnesium, ammonium, copper, or iron.

22. The method of claim 12, wherein said chemicals are hydrogen peroxide, sodium hydroxide and ethyl acetate.

23. The method of claim 12, wherein said conduits or wellbores are arranged in an array of patterns or configurations to displace injected chemicals into the subterranean formation and recover solubilized carbonaceous material.

24. The method of claim 17, wherein the solubilized carbonaceous material is introduced into an anaerobic fermentation system or systems of varying configuration selected from one-stage and two-stage fermentation systems.

25. The method according to claim 12, wherein the solubilization chemicals are introduced into the subterranean formation at a temperature of 10° C. to 250° C.

26. The method of claim 12, wherein the solubilizing chemicals are introduced into the subterranean formation under a pressure of 0 psig to 5000 psig per foot of depth from the surface to the depth of the subterranean formation.

27. The method of claim 12, wherein the subterranean formation is sonicated before or during introduction of said chemicals of step (b).

28. The method of claim 12, wherein said solubilization chemical is an acetate.

29. The method of claim 28, wherein said acetate is a member selected from the group consisting of methyl acetate, ethyl acetate, propyl acetate, isopropyl acetate, n-butyl acetate, isobutyl acetate, amyl acetate, isoamyl acetate, hexyl acetate, heptyl acetate, octyl acetate, nonanyl acetate, decyl acetate, undecyl acetate, lauryl acetate, tridecyl acetate, myristyl acetate, pentadecyl acetate, cetyl acetate, heptadecyl acetate, stearyl acetate, behenyl acetate, hexacosyl acetate, triacontyl acetate, benzyl acetate, bornyl acetate, isobornyl acetate and cyclohexyl acetate.

30. The method of claim 12, wherein the carbonaceous material is coal.

31. The method of claim 30, wherein the coal is selected from the group consisting of lignite, brown coal, sub-bituminous coal, bituminous coal, anthracite, and combinations thereof.

32. The method of claim 12, wherein said contacting further includes contacting with a solvent selected from the group consisting of phenanthrene, chrysene, fluoranthene and pyrene, a nitrogenous ring aromatic, anthracene, fluorene and combinations of any of these.

33. The method of claim 12, wherein said contacting further includes contacting with a solvent selected from the group consisting of phosphorous acid, phosphoric acid, a phosphite ester, triethylamine, quinuclidine HCl, pyridine, acetonitrile, diethylether, acetone, dimethyl acetamide, dimethyl sulfoxide, tetrahydrothiophene, trimethylphosphine, HNO3, EDTA, sodium salicylate, triethanolamine, 1,10-o-phenanthroline, sodium acetate, ammonium tartrate, ammonium oxalate, ammonium citrate tribasic, 2,3-dihydroxylbenzoic acid, 2,4-dihydroxylbenzoic acid, 3,4-dihydroxylbenzoic acid, 3,5-dihydroxylbenzoic acid, THF—tetrahydrofuran.

34. The method of claim 12, further comprising the step of contacting said solubilized carbonaceous material in situ with an anaerobic fermentation system for bioconversion of said solubilized carbonaceous material into a gas.

35. A bioconversion method comprising:
contacting a solubilized carbonaceous material with a bioconversion agent to bioconvert said material.

36. The method of claim 35, wherein the carbonaceous material is coal.

37. The method of claim 36, wherein the coal is selected from the group consisting of lignite, brown coal, sub-bituminous coal, bituminous coal, anthracite, and combinations thereof.

38. The method of claim 35, wherein said contacting occurs in situ.

39. The method of claim 35, wherein said contacting occurs ex situ.

40. A method of treating a carbonaceous material, comprising:
contacting a carbonaceous material with one or more chemicals selected from a peroxide, a hydroxide, and an ester of a C1-C4 carboxylic acid or a benzoic acid, thereby solubilizing at least a portion of the carbonaceous material.

41. The method of claim 40, wherein the carbonaceous material is coal.

42. The method of claim 40, wherein the coal is selected from the group consisting of lignite, brown coal, sub-bituminous coal, bituminous coal, anthracite, and combinations thereof.

43. A composition comprising the solubilized carbonaceous material formed by contacting a carbonaceous material with one or more chemicals selected from a peroxide, a hydroxide, and an ester of a C1-C4 carboxylic acid or a benzoic acid, thereby solubilizing at least a portion of the carbonaceous material.

44. The composition of claim 43, wherein the carbonaceous material is coal.

45. The composition of claim 43, wherein the coal is selected from the group consisting of lignite, brown coal, sub-bituminous coal, bituminous coal, anthracite, and combinations thereof.
